(19) United States
(12) Patent Application Publication
(10) Pub. No.: US 2025/0272875 A1
(43) Pub. Date: Aug. 28, 2025

(54) METHOD FOR CARRYING OUT PATIENT REGISTRATION ON A MEDICAL VISUALIZATION SYSTEM, AND MEDICAL VISUALIZATION SYSTEM

(71) Applicant: CARL ZEISS MEDITEC AG, Jena (DE)

(72) Inventors: Richard BAEUMER, Urbach (DE); Johannes RANGEL, Aalen (DE)

(21) Appl. No.: 19/058,182

(22) Filed: Feb. 20, 2025

(30) Foreign Application Priority Data
Feb. 22, 2024 (DE) 10 2024 201 660.8

(51) Int. Cl. G06T 7/73; G16H 10/60; G16H 30/20

(52) U.S. Cl. CPC G06T 7/74; G16H 10/60; G16H 30/20

(57) ABSTRACT

A method for carrying out patient registration on a medical visualization system includes performing an initial patient registration, wherein parameters of a transformation rule between a reference coordinate system and a patient coordinate system are determined, and also, for at least one camera pose of a main camera of the medical visualization system, the method includes determining the camera pose in the reference coordinate system, capturing a patient image using the main camera, and at least once generating a two-dimensional projection image of preoperatively acquired three-dimensional patient data based on the determined camera pose of the main camera, taking into account the transformation rule, and minimizing a deviation between the captured patient image and the generated two-dimensional projection image corresponding thereto by adapting parameters of the transformation rule. The method includes providing the adapted transformation rule.

The invention relates to a method for carrying out patient registration on a medical visualization system and to a medical visualization system.

In order to use preoperatively acquired data, such as for example computed tomography (CT) data or magnetic resonance tomography (MRT) data, in surgery, it is necessary to know a relationship between a reference coordinate system and a patient or patient coordinate system during the operation. Determining this relationship or a transformation rule, describing the relationship, between the coordinate systems is usually performed prior to the operation and is known as patient registration. The patient registration comprises in particular determining the relative pose (translation and rotation) of a patient in another coordinate system that does not move relative to the patient and therefore serves as reference coordinate system.

To register the patient, a surface of the patient and a pose of a reference object localized in the reference coordinate system are determined, for example by way of a unique tracker or a unique marker that is affixed to the patient (for example a Mayfield clamp). The surface of the patient may be determined in the reference coordinate system for example by way of stereoscopic topography determination (for example derived from stereoscopic image data from a surgical microscope) or by way of scanning devices that operate in contact-based or contactless fashion, such as for example a Brainlab Softtouch or a Brainlab Z-Touch (both from Brainlab AG). The pose of the reference object (of the marker or tracker on the patient) may be acquired and determined using a navigation system (for example from NDI, Canada) or, alternatively, using an internal tracking system of a medical visualization system.

The accuracy of the patient registration depends largely on the determination of the surface (topography, Softtouch or Z-touch) of the patient. In the case of topography determination using stereoscopic image data, very accurate camera calibrations are required. Furthermore, a stereo base in surgical microscopes at a working distance of typically 200 mm to 650 mm is small (in particular between 2 and 3 cm), which especially makes the estimation of the depth of the patient in the camera system of the medical visualization system inaccurate. It is therefore desirable to improve the accuracy of patient registration.

EP 1 142 536 A1 has disclosed a method for referencing a patient or a body part of a patient in a camera-assisted, medical navigation system, including the following steps: the body part of the patient to be referenced is brought into the capture region of a navigation system assisted by at least two cameras, with the navigation system capturing the three-dimensional spatial positions of light markings with computer assistance, a light beam is used to generate light markings on the surface of the body part to be referenced, with the three-dimensional position of said light markings being determined by the camera-assisted navigation system, the spatial pose of the surface of the body part to be referenced is determined using the position data for the light markings.

J. Kang, W. Liu, W. Tu and L. Yang, YOLO-6D+: Single Shot 6D Pose Estimation Using Privileged Silhouette Information, 2020 International Conference on Image Processing and Robotics (ICIP), Negombo, Sri Lanka, 2020, pp. 1-6, doi: 10.1109/ICIP48927.2020.9367354, describes a pose estimation method.

The invention is based on the object of improving a method for carrying out patient registration on a medical visualization system and a medical visualization system, in particular with regard to patient registration accuracy.

According to the invention, the object is achieved by a method having the features of patent claim 1 and a medical visualization system having the features of patent claim 14. Advantageous embodiments of the invention are evident from the dependent claims.

One of the core concepts of the invention, proceeding from an initially performed patient registration, is that of improving a transformation rule between a reference coordinate system and a patient coordinate system with regard to accuracy by performing an optimization based on a deviation between two-dimensional images. The idea behind this is that a main camera of the medical visualization system uses the degrees of freedom whose respective value is able to be estimated with greater accuracy to optimize the transformation rule. These degrees of freedom are the degrees of freedom that lie in the image plane of an image of the main camera, namely the x-direction, the y-direction and the rotation (in particular roll angle of the main camera about an optical axis) with respect to the image or the image plane (plane of an image sensor of the main camera). Values of the other degrees of freedom, such as the z-direction along the optical axis of the main camera and the other directions of rotation (in particular yaw and pitch angles of the main camera), are not able to be estimated as well from a captured image, and are therefore not used. The accuracy of the transformation rule is improved in that a patient image is captured for at least one camera pose, preferably at least two or more camera poses, of the main camera. A two-dimensional projection image is also generated for the same camera pose based on preoperatively acquired three-dimensional patient data (for example CT or MRT data, etc.). The two-dimensional projection image is generated in this case in particular such that it is arranged at the same position and in the same orientation and scale in the reference coordinate system with reference to the captured patient image. The two-dimensional projection image is generated in particular by way of projecting the preoperatively acquired three-dimensional patient data, in accordance with the laws of geometric optics, onto a plane of an image sensor of the main camera. The pose of the image sensor may be derived in this case in particular from the camera pose (taking into account an intrinsic and extrinsic calibration). Ideally, that is to say if the transformation rule were to be error-free, the captured (two-dimensional) patient image and the two-dimensional projection image corresponding thereto via the camera pose would have to be congruent, that is to say in particular have the same image content or comprise the same image section. In reality, however, there will be a deviation if the transformation rule is incorrect. To minimize this deviation, parameters of the transformation rule are adapted based on the captured patient image and the generated two-dimensional projection image. The deviation is determined and minimized in this case in particular based on the captured two-dimensional patient image and the generated two-dimensional projection image. At the end of the method, it is then possible to provide a transformation rule that is improved in terms of accuracy.

Provision is made in particular for a method for carrying out patient registration on a medical visualization system, comprising: performing an initial patient registration, wherein parameters of a transformation rule between a reference coordinate system and a patient coordinate system are determined, and also, for at least one camera pose of a main camera of the medical visualization system:
determining the camera pose in the reference coordinate system,
capturing a patient image using the main camera, and
at least once:
   generating a two-dimensional projection image of preoperatively acquired three-dimensional patient data based on the determined camera pose of the main camera, taking into account the transformation rule, and
   minimizing a deviation between the captured patient image and the generated two-dimensional projection image corresponding thereto by adapting parameters of the transformation rule; and
      providing the adapted transformation rule.

Provision is furthermore made in particular for a medical visualization system, comprising a main camera and a control device, wherein the control device is configured to obtain and/or determine parameters of a transformation rule between a reference coordinate system and a patient coordinate system as part of an initial patient registration, and, for at least one camera pose of the main camera:
to obtain a camera pose determined in the reference coordinate system or to determine the camera pose,
to initiate the capture of a patient image in the at least one camera pose by way of the main camera and/or to obtain a patient image captured in the at least one camera pose, and, at least once:
   to generate a two-dimensional projection image of preoperatively acquired three-dimensional patient data based on the determined camera pose of the main camera, taking into account the transformation rule, and
   to minimize a deviation between the captured patient image and the generated two-dimensional projection image corresponding thereto by adapting parameters of the transformation rule; and
      to provide the adapted transformation rule.

One advantage of the method and of the medical visualization system is that the transformation rule is adapted and/or optimized based on the degrees of freedom in the images of the main camera whose values are able to be determined most accurately with respect to a deviation. These are in particular the degrees of freedom that correspond to the two-dimensional image element plane of an image sensor of the main camera. These are in particular the degrees of freedom that correspond to the coordinate axes of the image element sensor and a rotation of the image element sensor about a surface normal running perpendicularly through the plane of the image element sensor (that is to say the x-direction, y-direction and rotation degrees of freedom).

The medical visualization system is in particular a surgical microscope. However, the medical visualization system may also be a microscope used for medical examinations and/or for diagnostic purposes. The method may in principle be used in particular in microscopes that operate with high magnification and in which, based thereon, there are three out of six degrees of freedom whose values are able to be determined more accurately in a captured image from the main camera than those of the other three degrees of freedom.

The two-dimensional projection image is determined based on the respective camera pose of the main camera. For this purpose, in particular, a known extrinsic and intrinsic calibration of the main camera, which may be determined in a manner known per se, are taken into account. The intrinsic calibration in this case describes in particular properties or spatial relationships of an imaging optical unit of the main camera. Properties are for example a description of distortions or a focal length. The intrinsic calibration parameters make it possible to image an object whose position in the coordinate system of the main camera is known onto the corresponding sensor of the main camera. The extrinsic calibration describes in particular a relative spatial relationship of the main camera with respect to the reference coordinate system or with respect to a tracking system operating in the reference coordinate system (for example a surroundings camera). With the aid of the known camera pose in which the patient image was also captured, it is possible, using the transformation rule (and the known intrinsic and extrinsic calibration), to establish a spatial relationship with respect to the patient coordinate system in which the preoperatively acquired three-dimensional patient data are present. These preoperatively acquired three-dimensional patient data are projected into the image plane or image sensor plane of the main camera by way of relationships and methods that are known per se in order to obtain the two-dimensional projection image. By way of example, for this purpose, it is possible to use a pinhole camera model for the main camera. A point from the 3D image space (X_{w}, Y_{w}, Z_{w}) may be transformed into the image space (u, v) by way of the pinhole camera model. This is described by way of example for the OpenCV software package at https://docs.opencv.org/4.x/d9/doc/group_calib3d.html. The generated two-dimensional projection image has in particular the same number of image elements and the same width and height, that is to say in particular the same image format, as the captured patient image. If the number of image elements and/or the format is different, appropriate scaling is carried out in order to be able to compare the images directly with one another.

In particular, as part of the method, a deviation between the captured patient image and the projection image corresponding thereto via the camera pose is determined and minimized. Adapting the parameters in particular causes a subsequently generated two-dimensional projection image to change, such that the deviation in relation to the captured patient image is able to be influenced and thus minimized, in particular step-by-step.

Using the transformation rule, coordinates of the reference coordinate system are able to be converted into coordinates of the patient coordinate system and vice versa. Using the transformation rule, the preoperatively acquired three-dimensional patient data are able to be localized in the reference coordinate system. Using the camera pose, which is likewise known in the reference coordinate system, the two-dimensional projection image is able to be generated based on the preoperatively acquired three-dimensional patient data arranged in this way, said two-dimensional projection image ideally, that is to say in the case of an error-free transformation rule, being congruent with the patient image captured in this camera pose. The deviation between the images may be used to quantify a magnitude of an error in the real case of an erroneous transformation rule, and the magnitude of the error may be used to correct the parameters of the transformation rule. The relationship between the patient coordinate system and the reference coordinate system, that is to say the transformation rule, may be described via a 6D pose. This may be done for example via a 3×1 rotation vector and a 3×1 translation vector, or alternatively via quaternions. Assuming a description is given of a point P with a normal to the surface in a coordinate system K₁, then its position P₁ and orientation R₁ may be represented as follows:

P₁=[R₁P₁;0 0 0 1]

wherein R₁ has the dimension 3×3 and P₁ has the dimension 3×1. Using a transformation matrix with the rotation R₁₂ (dimension 3×3) and the translation T₁₂ (dimension 3×1), the description of the point P may be converted from the coordinate system K₁ to a coordinate system K₂.

P₂=[R₁₂ T₁₂;0 0 0 1]*[R₁ P₁;0 0 0 1]

As an alternative, a point P in a coordinate system may be described with its three coordinates (x,y,z). Using a transformation rule, this may be transformed into another coordinate system K₂.

[P_{x,2};P_{y,2};P_{z,2};1]=[R₁₂ T₁₂;0 0 0 1]*[P_{x,1};P_{y,1};P_{z,1};1]

[P_{x,2};P_{y,2};P_{z,2};1] describes the point P in the second coordinate system K₂, [P_{x,1};P_{y,1};P_{z,1};1] the point P in the coordinate system K₁ and the 4×4 matrix [R₁₂ T₁₂;0 0 0 1] is the transformation matrix from the coordinate system K₂ to the coordinate system K₁.

The method makes it possible to take into account the entire transformation chain between the reference coordinate system and the patient coordinate system. In particular, in this case, errors in a tracking system and an intrinsic and extrinsic calibration may be compensated for by virtue of these initially defined calibration parameters also being reactivated (being able to be optimized).

As part of the initial patient registration, the transformation rule between a reference coordinate system and a patient coordinate system is estimated. This estimate forms a starting point for the optimization performed using the method in order to increase the accuracy of the transformation rule. The initial patient registration may be carried out in various ways. The initial patient registration may be carried out in particular using methods that are known per se. Some other exemplary options for the initial patient registration will be described later in this disclosure.

Provision may be made for at least one further main camera, for example as part of a stereo camera of the medical visualization system. The method may then additionally also be performed for the at least one further main camera, wherein the procedure here is basically the same as in the case of the main camera.

After performing the minimization, the adapted transformation rule is provided. The provision may in particular comprise loading the adapted parameters of the transformation rule into a memory of the control device so that the adapted transformation rule is available for subsequent use. In particular, the preoperatively acquired three-dimensional patient data may be placed over captured images that are captured by the main camera in order to augment them.

Parts of the medical visualization system, in particular the control device, may be designed, either individually or together, as a combination of hardware and software, for example as program code that is executed on a microcontroller or microprocessor. However, provision may also be made for parts to be designed, either individually or together, as an application-specific integrated circuit (ASIC) and/or a field-programmable gate array (FPGA). In particular, the control device comprises at least one computing device and at least one memory. The control device may furthermore comprise a communication interface for communication.

The method and the medical visualization system may be used in particular in the following fields: neurosurgery, spinal surgery, dental surgery, eye surgery, etc.

Provision is made in particular for the generation of the two-dimensional projection image and the minimization to be performed for at least one further camera pose. The camera poses in particular differ from one another (for example a different perspective, different acquisition angle, etc.). In particular, all six degrees of freedom of the medical visualization system may thereby be taken into account when minimizing the deviation. The transformation rule is then optimized or improved, in particular with regard to all six degrees of freedom. In other words, a respective patient image is captured in particular in at least two camera poses. In particular, a respective two-dimensional projection image is generated for each of the patient images. The deviation is then minimized taking into account the patient images and the associated two-dimensional projection images of the at least two camera poses.

In one embodiment, provision is made for features in the captured patient image and the two-dimensional projection image corresponding thereto to be recognized, wherein the deviation is minimized on the basis of the recognized features. This makes it possible to determine a deviation directly on the basis of features corresponding to one another. By way of example, features such as for example edges, contours, patterns, silhouettes, shadows and the like may be recognized in the images. Pattern recognition methods known per se from the fields of computer vision and/or machine learning may be used to recognize the features. A deviation between the images is able to be quantified on the basis of the recognized features. By way of example, provision may be made to determine a distance between features that correspond to one another in the images. A translation (in particular an x-direction and/or a y-direction of the image plane or of an image sensor) and a rotation (rotation angle) may be taken into account here. By way of example, the distance may be expressed in the form of a number of image elements. In the case of multiple features, a cumulative distance may be determined as a measure of a cumulative deviation. The deviation or cumulative deviation is minimized as part of the method by adapting the parameters of the transformation rule (step-by-step or iteratively).

In one embodiment, provision is made for the camera pose to be determined by way of a surroundings camera, used as internal tracking system, of the medical visualization system and at least one marker (for example a Mayfield clamp) arranged on the patient. This makes it possible to dispense with an external tracking system. It is assumed here that a spatial relationship between the surroundings camera and the main camera is known (that is to say in particular an extrinsic calibration is known).

In one embodiment, provision is made for the camera pose to be determined by way of an external tracking system and at least one marker (for example a Mayfield clamp) arranged on the patient and at least one marker arranged on the main camera. This makes it possible to determine the poses of the markers on the patient and on the main camera directly. The external tracking system then defines the reference coordinate system.

In one embodiment, provision is made for the reference coordinate system to be a coordinate system of a robotic stand, wherein the camera pose is determined in the reference coordinate system based on position data of an actuator system of the robotic stand. This has the advantage that no optical tracking is necessary. A relative starting pose of the robotic stand with respect to the patient may be estimated by way of image-based pose estimation. For this purpose, for example, a focal point of the main camera of the medical visualization system may be used for the initial position of the patient. A camera pose may be determined in the coordinate system of the robotic stand by way of a kinematic model, using which the camera pose is able to be calculated based on settings of actuators (axes of rotation and/or linear axes) of the robotic stand. By way of a further transformation that takes the optical image into account, it is then possible to determine the position of the focal point of the main camera in the coordinate system of the robotic stand (this is essentially a translation of the camera pose). It may in particular be assumed here that the focal point, when it is located at an excellent position on the patient, defines the patient coordinate system or coincides with an origin of the patient coordinate system. The excellent point is chosen in particular here such that it initially defines the origin of the patient coordinate system. The coordinate system of the robotic stand itself may be defined as a reference coordinate system, for example, with reference to an excellent point at a base of the robotic stand. The main camera is arranged in particular at a distal end of the robotic stand. The camera pose may in particular be changed and/or set by way of the robotic stand. The position data are acquired in particular with the aid of sensors arranged on articulations of the robotic stand corresponding to the respective actuator system. Such sensors may for example be encoders that encode respective rotation angles of articulations of the robotic stand. Based on this, it is possible to calculate a camera pose by way of a forward kinematics model.

Provision may be made for the camera pose to be determined by way of an internal tracking system and/or by way of an external tracking system and/or by way of a robotic stand. By combining different methods, the camera pose is able to be determined more accurately. Based on the individual methods, certain values for the camera pose are then provided, in particular in averaged form. Provision may in particular also be made for the camera pose to be determined by way of an internal tracking system and by way of an external tracking system and by way of a robotic stand.

In one embodiment, provision is made for the initial patient registration to be carried out by capturing at least one selected region of a patient by way of a capture device in a reference coordinate system and adapting the preoperatively acquired three-dimensional patient data, which are present with reference to a patient coordinate system, to the captured region, and determining parameters of the transformation rule between the reference coordinate system and the patient coordinate system. The initial patient registration is then carried out in particular using methods that are known per se from the prior art. The capture device may for example be a capture device that operates in contact-based or contactless fashion and that is configured to capture the selected region of the patient with regard to its spatial properties (for example surface profile, contour, etc.). The capture device may also be part of the medical visualization system or comprise at least part of the medical visualization system.

In one embodiment, provision is made for the initial patient registration to comprise capturing and/or deriving a surface contour of at least the selected region, wherein the adaptation to the preoperatively acquired three-dimensional patient data takes place based on the captured and/or derived surface contour. This makes it possible already to achieve high accuracy for the initial patient registration. To capture the surface contour, the patient (or the selected region) may be captured monoscopically or stereoscopically, for example using a stereo camera. A marker (tracker, for example a Mayfield clamp) arranged fixedly on the patient may in particular also be captured here, such that it is possible to acquire a pose of the surface contour in the reference coordinate system defined by the marker. As an alternative or in addition, the surface contour may be determined by way of a scanning device (capture device) that operates in contact-based or contactless fashion, such as for example a Brainlab Softtouch or a Brainlab Z-Touch. Such scanning devices usually have markers that are able to be captured and localized in the reference coordinate system by way of an internal tracking system (for example surroundings camera of the medical visualization system) or an external tracking system (navigation system), such that it is possible to acquire a pose of the scanning device in the reference coordinate system. A marker arranged fixedly on the patient may also be captured.

In one embodiment, provision is made for the initial patient registration to comprise carrying out a pose estimation of the main camera with respect to the patient. The pose estimation may be carried out here in particular with respect to a coordinate system of a robotic stand, used as reference coordinate system. Based on the estimated pose of the main camera with respect to the patient, it is possible to determine the pose of the patient in the reference coordinate system. The reference coordinate system may in particular be the coordinate system of the robotic stand. The camera pose may be determined by way of a kinematic model or a transformation derived therefrom in the coordinate system of the robotic stand, and thus in the reference coordinate system. Based on this, it is possible to determine the transformation rule between the reference coordinate system and the patient coordinate system via the estimated pose of the main camera with respect to the patient. The pose estimation may be carried out for example by way of a method as described in J. Kang, W. Liu, W. Tu and L. Yang, YOLO-6D+: Single Shot 6D Pose Estimation Using Privileged Silhouette Information, 2020 International Conference on Image Processing and Robotics (ICIP), Negombo, Sri Lanka, 2020, pp. 1-6, doi: 10.1109/ICIP48927.2020.9367354.

In one embodiment, provision is made, for the initial patient registration, for a captured patient image to be displayed in a manner superimposed with a projection image generated based on the preoperatively acquired patient data, and for the main camera to be arranged in a target camera pose by a user on the basis of the superimposed images. The initial patient registration is thereby able to be performed with the aid of a user. Provision may for example be made, depending on a type of planned operation, for a suitable projection image to be generated based on patient data acquired preoperatively for the preparation of the operation. As a rule, a surgical intervention is carried out from a predefined direction. By way of example, for this direction, the projection image is generated from the preoperatively acquired patient data and, for example, displayed on a display device of the medical visualization system. At the same time, a patient image is captured by the patient using the main camera and likewise displayed on the display device. The projection image is in particular displayed in a manner superimposed with the patient image here. Provision is made in particular for patient images to be captured continuously or repeatedly by way of the main camera, in particular in the form of a real-time video in which a change in the arrangement of the main camera appears immediately. A user may then adapt the pose of the main camera based on the superimposed representation of the images such that the projection image and the patient image comprise the same content (for example part of the head, etc.). If the images are congruent in a camera pose, then the associated camera pose is determined. If the reference coordinate system is a coordinate system of a robotic stand, then the coordinates of the camera pose may be determined for example based on sensor values of an actuator system. The transformation rule for the initial patient registration is determined based on the determined camera pose.

In one embodiment, provision is made for a set of camera poses to be provided, wherein the minimization is performed based on all captured patient images and generated two-dimensional projection images, respectively corresponding thereto, of the set. This makes it possible to further improve the accuracy of the transformation rule. In particular, as part of the method, an overall deviation for the set is determined and minimized.

In one embodiment, provision is made for markings to be arranged and/or for selected regions to be identified on the patient, wherein the deviation is minimized taking into account the markings and/or the selected regions. This makes it possible, in a targeted manner, to mark and/or identify regions of the patient that may be used when determining the deviation. Provision is in particular made here for the markings to be used and/or captured (or registered) as part of the initial patient registration.

In one embodiment, provision is made for at least one region in which the patient is arranged to be structurally illuminated during the capture of the patient image, wherein the deviation is minimized taking into account the structural illumination. This makes it possible to assist the determination of the deviation. For this purpose, it is necessary to know in particular a pose of an illumination unit that generates the structural illumination with respect to the main camera. This may be known in advance or ascertained through extrinsic calibration. It is also necessary to know a direction of light beams of the structural illumination emanating from the illumination unit. The light beams from the illumination unit, in this embodiment, have to be taken into account accordingly in the generated two-dimensional projection image via the known direction. In other words, the structural illumination is also generated in the projection image based on the known direction of the light beams. When working for example with linear projection, the topography of the patient both in the captured patient image and in the generated two-dimensional projection image causes the lines of the projection to become curves that are able to be recognized and assigned to one another. The transformation rule is then optimized in particular by minimizing a deviation between the poses or positions of the curves in the superimposed images. As an alternative, it is possible to work with other projection patterns or lasers, with the procedure being the same.

In one embodiment, provision is made for the at least one camera pose of the main camera to be set automatically by way of a robotic stand. Different camera poses are thereby able to be set automatically. This in particular allows fully automated capturing of the patient image in multiple camera poses. The method may thereby be performed automatically at the use location of the medical visualization system without any great outlay in terms of personnel and without special training.

Further features relating to the design of the medical visualization system will become apparent from the description of embodiments of the method. The advantages of the medical visualization system here are in each case the same as for the embodiments of the method.

The invention is explained in greater detail below on the basis of preferred exemplary embodiments with reference to the figures. In the figures:

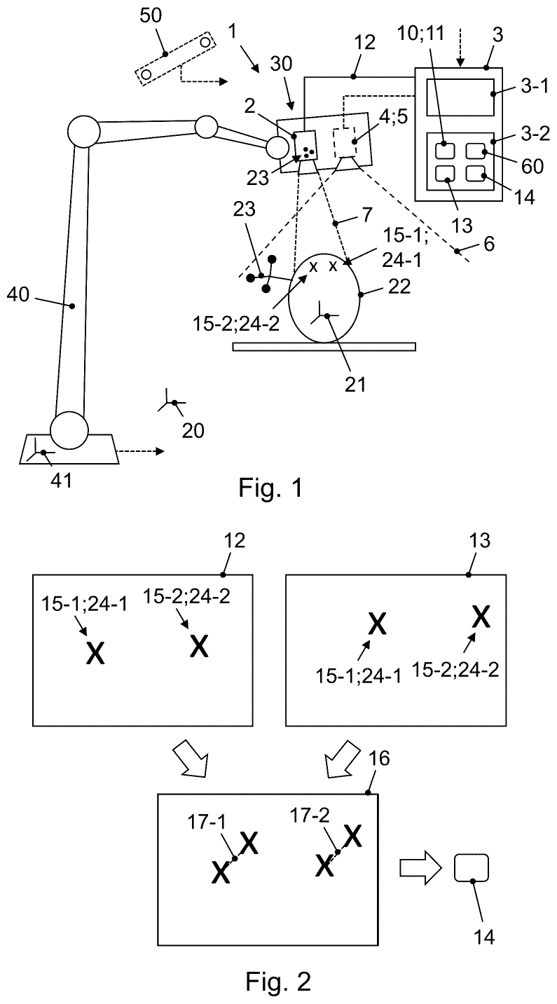

FIG. 1 shows a schematic illustration for the purpose of illustrating embodiments of the medical visualization system;

FIG. 2 shows a schematic illustration for the purpose of illustrating the determination of the deviation;

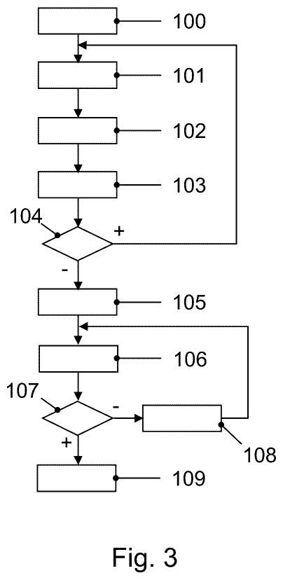

FIG. 3 shows a schematic flowchart for the purpose of illustrating embodiments of the method.

FIG. 1 shows a schematic illustration for the purpose of illustrating embodiments of the medical visualization system 1 and the method. The medical visualization system 1 comprises a main camera 2 and a control device 3. The control device 3 comprises a computing device 3-1 and a memory 3-2.

Provision may be made for at least one further main camera (not shown), for example as part of a stereo camera. The method may additionally also be performed with the aid of at least one further main camera, with the procedure here being the same as when using the main camera. The respective camera pose of at least one further main camera is then taken into account accordingly.

The control device 3 is configured to obtain and/or determine parameters 11 of a transformation rule 10 between a reference coordinate system 20 and a patient coordinate system 21 as part of an initial patient registration. The medical visualization system 1 comprises in particular a robotic stand 40 in the embodiment shown.

Provision may be made for the initial patient registration to be carried out by capturing at least one selected region of a patient 22 by way of a capture device in a reference coordinate system 20 and adapting preoperatively acquired three-dimensional patient data 60, which are present with reference to a patient coordinate system 21, to the captured region, and determining parameters of the transformation rule 10 between the reference coordinate system 20 and the patient coordinate system 21. The capture device may be for example the main camera 2.

The control device 3 is furthermore configured, for at least one camera pose 30 of the main camera 2, to obtain a camera pose 30 determined in the reference coordinate system 20 or to determine the camera pose 30, to initiate the capture of a patient image 12 in the at least one camera pose 30 by way of the main camera 2 and/or to obtain a patient image 12 captured in the at least one camera pose 30.

Furthermore, a two-dimensional projection image 13 of the preoperatively acquired three-dimensional patient data 60 is generated at least once by way of the control device 3 based on the determined camera pose 30 of the main camera 2 taking into account the transformation rule 10, and a deviation 14 between the captured patient image 12 and the generated two-dimensional projection image 13 corresponding thereto is minimized by adapting the parameters 11 of the transformation rule 10. In particular, for this purpose, a two-dimensional projection image 13 is generated repeatedly and/or iteratively, taking into account the adapted transformation rule 10, and a deviation 14 is again determined in order to adapt the parameters 11 again. This is repeated in particular until the deviation 14 falls below a predefined threshold value.

The adapted transformation rule 10 is then provided. In particular, the provision may comprise loading the adapted transformation rule 10 into a memory 3-2 or memory area of the control device 3 in order to provide the transformation rule 10 subsequently for application. Such an application may include for example superimposing the preoperatively acquired patient data 60 with a patient image 12 captured by way of the main camera 2, in particular in order to augment the captured patient image 12.

FIG. 2 shows a schematic illustration for the purpose of illustrating the determination of the deviation 14 between the captured patient image 12 and the generated two-dimensional projection image 13. In the example shown, it is assumed that the transformation rule is incorrect, which ultimately means that the patient image 12 and the projection image 13 have different image content. In other words, the images 12, 13 show sections of the patient 22 that differ from one another, since the incorrect transformation rule leads to the coordinates of the camera pose in which the patient image 12 was captured being transformed incorrectly into the patient coordinate system in which the preoperatively acquired three-dimensional patient data 60 are present, and vice versa. As a result of this incorrect transformation, the projection image 13 generated based on the preoperatively acquired patient data in projection onto the camera pose shows a different image section than the acquired patient image 12. The same features 15-1, 15-2 in the images 12, 13, such as for example edges, contours or patterns, therefore have different positions in the images 12, 13 (described for example with reference to image element coordinates of an image sensor in the x-direction and y-direction). This is illustrated with reference to a superimposed image 16, which is likewise shown in FIG. 2. The images 12, 13 are superimposed on one another in this figure. It may be seen that the same features 15-1, 15-2 are at a respective distance 17-1, 17-2 from one another. This distance 17-1, 17-2 may be quantified for example in the coordinate system of the image elements. By adapting the parameters of the transformation rule, it is possible to reduce the respective distance 17-1, 17-2, such that the features 15-1, 15-2 ideally lie directly on top of one another, that is to say in particular each lie at the same image element coordinates. Provision may be made for example for a measure of the deviation 14 to be determined from the distances 17-1, 17-2, for example by forming a sum of the distances 17-1, 17-2 or another suitable measure (for example mean squared deviation, etc.). By adapting the parameters of the transformation step-by-step using optimization methods that are known per se and regenerating the two-dimensional projection image 13 using the transformation rule adapted in this way, the deviation 14 is able to be determined again. The deviation 14 may thereby be minimized step-by-step or iteratively.

Provision may be made in particular for the features 15-1, 15-2 in the captured patient image 12 and the two-dimensional projection image 13 corresponding thereto to be recognized, wherein the deviation 14 is minimized on the basis of the recognized features 15-1, 15-2. Computer vision and/or artificial intelligence methods that are known per se, such as machine learning and/or pattern recognition methods, may be used to recognize the features 15-1, 15-2.

Provision may be made for the camera pose 30 (FIG. 1) to be determined by way of a surroundings camera 5, used as internal tracking system 4, of the medical visualization system 1 and at least one marker 23 (tracker, for example a Mayfield clamp) arranged on the patient 22. The surroundings camera 5 has a capture region 6 that captures the patient 22 and the marker 23 and in particular also the surroundings of the patient 22. The capture region 6 of the surroundings camera 5 is generally significantly larger than a capture region 7 of the main camera 2.

Provision may be made for the camera pose 30 to be determined by way of an external tracking system 50 and at least one marker 23 arranged on the patient 22 and at least one marker 23 arranged on the main camera 2. By way of example, the external tracking system 50 may be a navigation system (for example from NDI, Canada).

Provision may be made for the reference coordinate system 20 to be a coordinate system 41 of a robotic stand 40, wherein the camera pose 30 is determined in the patient coordinate system 21 based on position data of an actuator system of the robotic stand 40.

Provision may be made for the initial patient registration to comprise capturing and/or deriving a surface contour of at least the selected region, wherein the adaptation to the preoperatively acquired three-dimensional patient data 60 takes place based on the captured and/or derived surface contour.

Provision may be made for the initial patient registration to comprise carrying out a pose estimation of the main camera 2 with respect to the patient 22. This may be carried out for example by way of the method described in J. Kang, W. Liu, W. Tu and L. Yang, YOLO-6D+: Single Shot 6D Pose Estimation Using Privileged Silhouette Information, 2020 International Conference on Image Processing and Robotics (ICIP), Negombo, Sri Lanka, 2020, pp. 1-6, doi: 10.1109/ICIP48927.2020.9367354. The transformation rule 10 is available based thereon and may then be optimized in accordance with the method.

Provision may also be made, for the initial patient registration, for a captured patient image 12 to be displayed in a manner superimposed with a projection image 13 generated based on the preoperatively acquired patient data 60, and for the main camera 2 to be arranged in a target camera pose by a user on the basis of the superimposed images 12, 13. By way of example, the display is carried out on a display device (not shown) of the medical visualization system.

Provision may be made for a set of camera poses 30 to be provided, wherein the minimization is performed based on all captured patient images 12 and generated two-dimensional projection images 13, respectively corresponding thereto, of the set. The set may in particular comprise at least two camera poses 30. Preferably, the set comprises further camera poses 30. For all image pairs—as explained by way of example with reference to FIG. 2—the respective deviation 14 is then determined and minimized by adapting the parameters of the transformation rule 10. In particular, a cumulative deviation 14 is determined and minimized for the entire set.

Provision may be made for markings 24-1, 24-2 to be arranged and/or for selected regions to be identified on the patient 22, wherein the deviation 14 is minimized taking into account the markings 24-1, 24-2 and/or the selected regions.

Provision may be made for at least one region in which the patient 22 is arranged to be structurally illuminated during the capture of the patient image 12, wherein the deviation 14 is minimized taking into account the structural illumination.

Provision may be made for the at least one camera pose 30 of the main camera 2 to be set automatically by way of the robotic stand 40. For this purpose, the robotic stand 40 is driven in particular by way of the control device 3. By way of example, provision may be made for two or three different camera poses 30 (for example via different angular positions of an axis of the robotic stand 40) of the main camera 2.

FIG. 3 shows a schematic flowchart for the purpose of illustrating embodiments of the method for carrying out patient registration on a medical visualization system. The medical visualization system may be designed for example like the medical visualization system described with reference to FIG. 1.

In a method step 100, an initial patient registration is carried out, for example by capturing at least one selected region of a patient by way of a capture device in a reference coordinate system and adapting preoperatively acquired three-dimensional patient data (for example CT or MRT), which are present with reference to a patient coordinate system, to the captured region, and determining parameters of a transformation rule between the reference coordinate system and the patient coordinate system. As an alternative or in addition, the initial patient registration may be performed in other ways.

In a method step 101, a camera pose of a main camera is set. This may be carried out automatically, for example, by way of a robotic stand or manually.

In a method step 102, the camera pose is determined in the reference coordinate system. This may be carried out by way of an internal tracking system (for example by way of a surroundings camera) or an external tracking system (for example navigation system).

In a method step 103, a patient image is acquired in the set camera pose by way of the main camera.

In a method step 104, a check is carried out to determine whether further camera poses are provided. If this is the case, the method returns to method step 101. If, on the other hand, this is not the case, that is to say if all provided camera poses have already been run through, the method continues with method step 105.

In method step 105, a respective two-dimensional projection image of the preoperatively acquired three-dimensional patient data is determined based on the respectively determined camera pose of the main camera, taking into account the transformation rule. For each captured patient image, a two-dimensional projection image corresponding thereto via the respective camera pose is then available. In total, a set of pairs each comprising a patient image paired with a projection image is thus available.

In a method step 106, a deviation is determined for each pair of acquired patient image and projection image corresponding respectively thereto. By way of example, feature recognition may be performed for this purpose, in the course of which features and/or contours etc. are recognized and used to determine the deviation. The deviation of the entire set is in particular a cumulative value of the deviations between the individual pairs within the set.

In method step 107, a check is carried out to determine whether the (cumulative) deviation is below a predefined threshold value. If this is the case, then the method continues with method step 109. If, on the other hand, this is not the case, then the method continues with step 108.

In method step 108, parameters of the transformation rule are adapted in order to minimize the deviation. Optimization methods that are known per se may be used here in order to determine the type and magnitude of the adaptation. The method then returns to method step 106, this being performed taking into account the adapted parameters of the transformation rule.

In method step 109, the adapted transformation rule is provided. For this purpose, the adapted transformation rule may be loaded for example into a memory or memory area of the medical visualization system. The preoperatively acquired patient data may then for example be superimposed with captured patient images for augmentation purposes and to assist a surgeon.

Further embodiments of the method have already been described with reference to the medical visualization system.

### LIST OF REFERENCE SIGNS

1 Medical visualization system
2 Main camera
3 Control device
3-1 Computing device
3-2 Memory
4 Internal tracking system
5 Surroundings camera
6 Capture region (surroundings camera)
7 Capture region (main camera)
10 Transformation rule
11 Parameters
12 Patient image
13 Projection image
14 Deviation
15-x Feature
16 Superimposed image
17-x Distance
20 Reference coordinate system
21 Patient coordinate system
22 Patient
23 Marker (tracker)
24-x Marking
30 Camera pose
40 Robotic stand
41 Coordinate system (robotic stand)
50 External tracking system
60 Preoperatively acquired three-dimensional patient data
100-109 Method steps

1. A method for carrying out patient registration on a medical visualization system, comprising:
performing an initial patient registration, wherein parameters of a transformation rule between a reference coordinate system and a patient coordinate system are determined, and also,
for at least one camera pose of a main camera of the medical visualization system:
determining the camera pose in the reference coordinate system,
capturing a patient image using the main camera, and
at least once:
generating a two-dimensional projection image of preoperatively acquired three-dimensional patient data based on the determined camera pose of the main camera, taking into account the transformation rule, and
minimizing a deviation between the captured patient image and the generated two-dimensional projection image corresponding thereto by adapting parameters of the transformation rule; and
providing the adapted transformation rule.

2. The method according to claim 1, wherein features in the captured patient image and the two-dimensional projection image corresponding thereto are recognized, wherein the deviation is minimized on the basis of the recognized features.

3. The method according to claim 1, wherein the camera pose is determined by way of a surroundings camera, used as internal tracking system, of the medical visualization system and at least one marker arranged on the patient.

4. The method according to claim 1, wherein the camera pose is determined by way of an external tracking system and at least one marker arranged on the patient and at least one marker arranged on the main camera.

5. The method according to claim 1, wherein the reference coordinate system is a coordinate system of a robotic stand, wherein the camera pose is determined in the reference coordinate system based on position data of an actuator system of the robotic stand.

6. The method according to claim 1, wherein the initial patient registration is carried out by capturing at least one selected region of a patient by way of a capture device in a reference coordinate system and adapting the preoperatively acquired three-dimensional patient data, which are present with reference to a patient coordinate system, to the captured region, and determining parameters of the transformation rule between the reference coordinate system and the patient coordinate system.

7. The method according to claim 6, wherein the initial patient registration comprises capturing and/or deriving a surface contour of at least the selected region, wherein the adaptation to the preoperatively acquired three-dimensional patient data takes place based on the captured and/or derived surface contour.

8. The method according to claim 1, wherein the initial patient registration comprises carrying out a pose estimation of the main camera with respect to the patient.

9. The method according to claim 1, wherein, for the initial patient registration, a captured patient image is displayed in a manner superimposed with a projection image generated based on the preoperatively acquired patient data, and the main camera is arranged in a target pose by a user on the basis of the superimposed images.

10. The method according to claim 1, wherein a set of camera poses is provided, wherein the minimization is performed based on all captured patient images and generated two-dimensional projection images, respectively corresponding thereto, of the set.

11. The method according to claim 1, wherein markings are arranged and/or selected regions are identified on the patient, wherein the deviation is minimized taking into account the markings and/or the selected regions.

12. The method according to claim 1, wherein at least one region in which the patient is arranged is structurally illuminated during the capture of the patient image, wherein the deviation is minimized taking into account the structural illumination.

13. The method according to claim 1, wherein the at least one camera pose of the main camera is set automatically by way of a robotic stand.

14. A medical visualization system, comprising:
a main camera,
a control device, wherein the control device is configured to obtain and/or determine parameters of a transformation rule between a reference coordinate system and a patient coordinate system as part of an initial patient registration, and
for at least one camera pose of the main camera:
to obtain a camera pose determined in the reference coordinate system or to determine the camera pose, to initiate the acquisition of a patient image in the at least one camera pose by way of the main camera and/or to obtain a patient image captured in the at least one camera pose, and, at least once:
to generate a two-dimensional projection image of preoperatively acquired three-dimensional patient data based on the determined camera pose of the main camera, taking into account the transformation rule, and
to minimize a deviation between the captured patient image and the generated two-dimensional projection image corresponding thereto by adapting parameters of the transformation rule; and
to provide the adapted transformation rule.
